(19) United States
(12) Patent Application Publication
(10) Pub. No.: US 2006/0100497 A1
(43) Pub. Date: May 11, 2006

(54) Medical information system, program product for use in the medical information system and method of processing data to manage a medical procedures

(76) Inventors: Hirokazu Sawazaki, Tokyo (JP); Yoichi Takada, Otawara-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/268,614

(22) Filed: Nov. 8, 2005

(30) Foreign Application Priority Data
Nov. 9, 2004 (JP) 2004-325509

(51) Int. Cl. A61B 5/05

(52) U.S. Cl. 600/407

(57) ABSTRACT

A medical information system includes a data-acquiring unit which acquires, in time sequence, condition data representing at least one of conditions of medical apparatuses used in a medical procedure, conditions of medical instruments used in the medical procedure, conditions of medical articles used in the medical procedure, conditions of patient and conditions of medical staff performing the medical procedure, a first determining unit which determines an order in which stages are performed, from a condition which is determined on the basis of the condition data and in which events occur, a second determining unit which determines a start timing of each of the stages on the basis of the condition data, and a unit which generates sequence data representing the order and the start timing of each stage.

### CROSS-REFERENCE TO RELATED APPLICATIONS

This application is based upon and claims the benefit of priority from prior Japanese Patent Application No. 2004-325509, filed Nov. 9, 2004, the entire contents of which are incorporated herein by reference.

### BACKGROUND OF THE INVENTION

1. Field of the Invention

The present invention relates to a medical information system that provides information useful in analyzing and verifying post-operation conditions, a program that enables computers to function as the medical information system, and a method of processing data to manage a medical procedures.

2. Description of the Related Art

Generally, after performing an operation on a patient, the surgeons and assistants who worked on the operations record the operation procedures in detail. The records are sometimes accompanied with charts, photographs and images that show data that various apparatuses output while the operation was undergoing.

In most cases, however, the various data items recorded are filed at random, not in chronological order. Even specialists, such as the surgeons, can hardly confirm or verify the operation procedure exactly, on the basis of the data items recorded.

In view of this it is proposed that video cameras be used to record the operation and that the sequence of the operation be confirmed and verified on the basis of the moving picture thus recorded. However, it takes the surgeon much time and labor to verify the operation procedure. This is because he or she must keep observing the moving picture, while analyzing each step of the procedure he or she has performed.

Jpn. Pat. Appln. KOKAI Publication No. 2002-253545 discloses the technique of recording the history of events, i.e., repeated switching from a tomogram to another, which is to be observed. Jpn. Pat. Appln. KOKAI Publication No. 2004-280455 discloses the technique of recording the history of events that takes place during an operation.

These techniques may be used, enabling the surgeon to confirm and verify the operation procedure he or she has performed. A great number of events occur during any operation. Hence, it takes the surgeon much time and labor to confirm and verify the operation procedure on the basis of the recorded history of events.

It has been difficult for any surgeon to confirm and verify, within a short time, the operation procedure he or she has performed.

### BRIEF SUMMARY OF THE INVENTION

In view of the foregoing, there is a demand for a technique that helps surgeons to confirm and verify easily the operation procedures they have performed.

According to a first aspect of the present invention, there is provided a medical information system for processing data to manage a medical procedures that includes a plurality of stages each of which is a combination of evens, the system comprising: a data-acquiring unit which acquires, in time sequence, condition data representing at least one of conditions of medical apparatuses used in the medical procedure, conditions of medical instruments used in the medical procedure, conditions of medical articles used in the medical procedure, conditions of patient and conditions of medical staff performing the medical procedure; a first determining unit which determines an order in which the stages are performed, from a condition which is determined on the basis of the condition data and in which the events occur; a second determining unit which determines a start timing of each of the stages on the basis of the condition data; and a unit which generates sequence data representing the order and the start timing of each stage.

According to a second aspect of the present invention, there is provided a program product for causing a computer to function as a medical information system designed to process data to manage medical procedures that include a plurality of stages each of which is a combination of evens, the system comprising: a data-acquiring unit which acquires, in time sequence, condition data representing at least one of conditions of medical apparatuses used in the medical procedure, conditions of medical instruments used in the medical procedure, conditions of medical articles used in the medical procedure, conditions of patient and conditions of medical staff performing the medical procedure; a first determining unit which determines an order in which the stages are performed, from a condition which is determined on the basis of the condition data and in which the events occur; a second determining unit which determines a start timing of each of the stages on the basis of the condition data; and a unit which generates sequence data representing the order and the start timing of each stage.

According to a third aspect of the present invention, there is provided a method of processing data to manage a medical procedures that includes a plurality of stages each of which is a combination of evens, the method comprising: acquiring, in time sequence, condition data representing at least one of conditions of medical apparatuses used in the medical procedure, conditions of medical instruments used in the medical procedure, conditions of medical articles used in the medical procedure, conditions of patient and conditions of medical staff performing the medical procedure; determining an order in which the stages are performed, from a condition which is determined on the basis of the condition data and in which the events occur; determining a start timing of each of the stages on the basis of the condition data; and

generating sequence data representing the order and the start timing of each stage.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram showing how a medical information system is used, which is embodiments of this invention;

FIG. 2 is a block diagram of the medical information system that is shown in FIG. 1;

FIG. 3 is a stage table that may be stored in the stage-table storage unit shown in FIG. 2 in a first embodiment of the invention;

FIG. 4 is a flowchart illustrating the sequence in which the procedure-analyzing unit shown in FIG. 2 generates the data representing a medical procedure that has been performed, in the first embodiment of the invention;

FIG. 5 is a diagram showing some data items that the procedure-analyzing unit shown in FIG. 2 has generated in the first embodiment;

FIG. 6 is a flowchart illustrating the sequence in which the procedure-analyzing unit shown in FIG. 2 generates the data representing a medical procedure that has been performed, in a second embodiment of this invention;

FIG. 7 is a diagram showing how events take place at the stage of stopping bleeding;

FIG. 8 is a diagram showing some data items that the procedure-analyzing unit shown in FIG. 2 has generated in the second embodiment; and

FIG. 9 is diagram showing some images that the display device shown in FIG. 2 displays in the second embodiment.

### DETAILED DESCRIPTION OF THE INVENTION

Embodiments of the present invention will be described, with reference to the accompanying drawings.

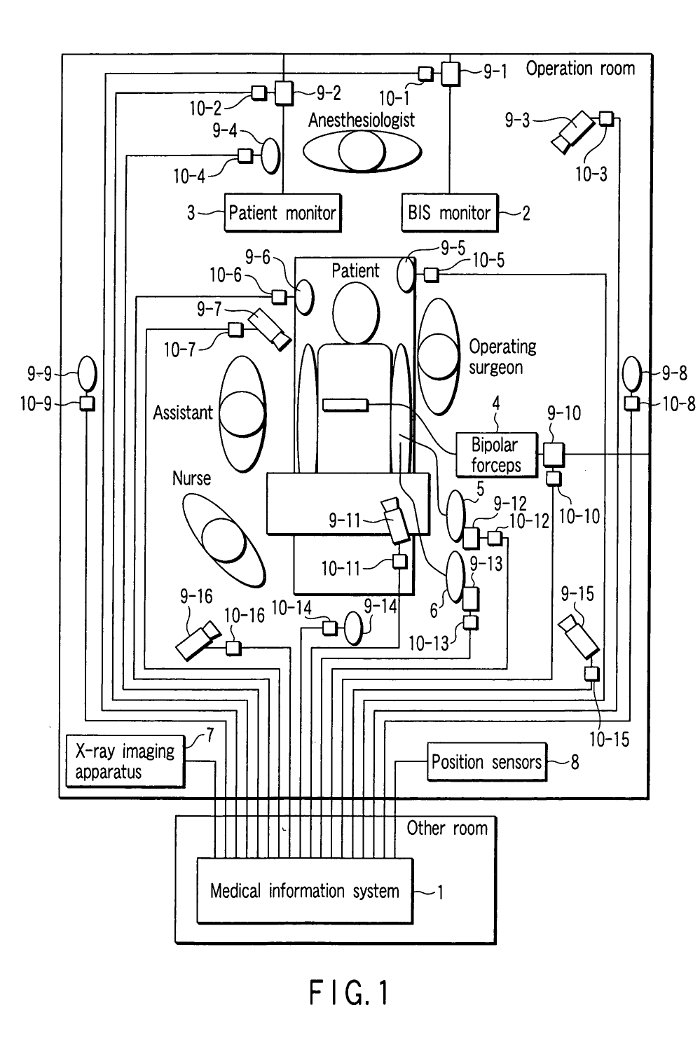

FIG. 1 shows how a medical information system 1 is used, which is an embodiment of this invention. The medical information system 1 is installed in a room other than the operation room, to acquire and record medical data about any operation performed in the operation room. The tools used in the operation include a BIS monitor 2, a patient monitor 3, a pair of bipolar forceps 4, a contrast-medium administering apparatus 5, a suction catheter 6 and an X-ray imaging apparatus 7.

The medical information system 1 records, in time sequence, almost all events taking place during medical procedures, such as operation, diagnosis, medicine administration, treatment, and rehabilitation. The system 1 automatically determines the sequence of any medical procedure, i.e., the sequence of stages. The medical data that is generated during an operation may include the activities of the medical staff, the motions they make, the positions they assume, the conditions of the patient, the conditions of the operation room, the operating conditions of the medical apparatuses, the positions of the apparatuses take, the positions of the medical instruments, the positions of the medical articles, the images of the region or tissues being treated, and the environmental conditions. In this embodiment, the term “medical procedure” means any medical sequence that is performed to complete operation, diagnosis, medicine administration, treatment or rehabilitation. The word “stage” means a medical process, or one of the steps that constitute a medical procedure. The word “event” means any activity that the surgeon or his or her assistant performs and any action that any apparatus used makes. Stages in, for example, coronary angiography, are: centesis of the femoral artery, insertion of a guide wire, insertion of a sheath, insertion of a catheter into the artery, insertion of a catheter into the coronary artery, angiography, and pressing. The centesis of the femoral artery includes events such as dermal incision and insertion of a centesis needle. Generally, several events constitute a stage, and several stages constitute a medical procedure.

Position sensors 8 and data-acquiring devices 9 are arranged in the operation room. The position sensors 8 and data-acquiring devices 9 collect items of medical data that should be recorded. Some of the position sensors 8 read ID data items from, for example, IC tags that lie near them. The ID data items are output to the medical information system 1. From the ID data items, the medical information system 1 determines that the positions of the IC tags that exist near the position sensors. Some of the IC tags are attached to the surgeon, the surgeon's assistants, the anesthesiologist and the nurses. The IC tags are attached to the medical apparatuses, the medical instruments and the medical articles that are movable.

The data-acquiring devices 9 acquire medical data other than the position data. The data-acquiring devices 9 can be classified into categories. The categories including first to seventh categories. The devices of the first category are the sensors provided on, or located near, the medical apparatuses, to obtain data presenting how the apparatuses are operating. The devices of the second category include cameras for capturing moving picture of several sites in the operation room and cameras for capturing moving picture of the region being treated. The devices of the third category are sensors for detecting the vitals of the surgeon and his or her assistants. The devices of the fourth category are microphones for detecting speech and sound made in the operation room. The devices of the fifth category are sensors for detecting the staff's coming into, and going out of, the operation room. The devices of the sixth category are sensors for acquiring the biological data about the patient. The devices of the seventh category are sensors for acquiring the ID data of the patient.

Some of the data-acquiring devices 9 will be described in detail, category by category, and the uses of their output signals will be explained.

(1) Devices of the First Category

a. Instillation-Monitoring Sensor

This sensor monitors the instillation and detects the pressure, flow rate and rate of dripping. It may determine the state of dripping from the rate at which the total weight of the bag and the solution decreases.

b. Bed-Angle Sensor

The sensor comprises an angle gauge and a pressure gauge that cooperate to measure the angle of the bed on which the patient lies.

c. Evacuation Sensor

The sensor detects the sound the evacuator makes, the pressure and rate at which the evacuator draws air, determines the operating state of the evacuator.

d. Electrode-Knife Sensor

This sensor infers how any electrode knife is driven, from the electric current supplied to the knife and the voltage applied thereto.

e. Bed-Warmer Temperature Sensor

The sensor measures the temperature of the bed warmer.

f. Sensors on the Anesthesia System

The sensors detect the density and amount of the anesthetic gas applied.

g. Sensor on the Navigator

This sensor detects the output and the like of the X-ray CT scanner.

h. Ground Current Sensor

The sensor measures the ground current of the operation room, thus monitoring the electric current flowing into the operation room.

i. Power-Supply Sensors

These sensors measures the intensities of light beams emitted from the pilot lamps on the medical apparatuses and determine, from the intensities of the beams, whether power is normally supplied to the medical apparatuses.

j. Sensors for the Dials and Switch of the Medical Apparatuses

The sensors may be angle monitors provided on the dials, cameras for capture moving picture of the dials (painted in different colors), contact switches, magnetic sensors, or optical sensors.

(2) Devices of the Second Category

a. Cameras Installed Near the Apparatus Tables

The cameras are installed near the tables on which instruments such as knifes are laid, to capture moving picture of the instruments being taken up from the tables.

b. Wide-Angle Camera

The camera for captures moving picture of the entire view of the operation room.

C. Operation-Field Camera

The camera (attached to a microscope) captures moving picture of the region being operated on. The camera may be attached to an endoscope.

d. Camera for the Anesthesiologist

This camera can scan the shelf holding medicines for emergency use (preferably, only when a change occurs on the shelf), and scan the screen of the monitor the anesthesiologist is observing. Otherwise, the camera may be one attached to the penlight the anesthesiologist uses.

e. Shadow-Less Camera

This is a combination of several cameras. Some of these cameras, which captured moving picture of an object well, are selected, and the outputs of the cameras selected are automatically output. Some cameras are selected in accordance with the brightness of image, the correctness of focal-distance adjustment (for auto-focusing cameras), the color of the caps the medical staff wear, the colors of markers, the distance measured by an ultrasonic distance sensor, the presence or absence of halation, the color code attached to the surgeon's cap, and the like.

f. Head-Mounted Camera

The camera is attached to the goggle or operating overall the surgeon wears and capture moving picture of any view field the surgeon's eyes can.

(3) Devices of the Third

a. Sensor for Detecting Biological Data

These sensors detect the heartbeats, adrenaline (stress hormone), brain waves, body temperature, blood pressure, and the like.

b. Blink Sensor

This is an optical sensor, an electromyography sensor or a camera. The optical sensor detects the positions of the eyelashes or the light beams reflected at the eyeballs as the operating surgeon or that detects. The electromyography sensor detects the motion of the eyelashes. The camera keeps capture moving picture of the operating surgeon's face as he or she looked up at the surgeon.

c. Acceleration Sensor

This sensor is attached to the surgeons arm. It is used as an inclination sensor, too. The sensor wires are provided on, for example, the surgeon's operating overall.

d. Perspiration Sensor

The sensor is designed to detect the patient's perspiration. It has electrodes, which are attached to the sock that the patient wears.

e. Pressure Sensor, Humidity Sensor, Respiration Sensor

These sensors are embedded in the operating mask that the surgeon wears.

(4) Devices of the Fourth Category

a. Microphone on the Operating Surgeon

The microphone records the voice and speech the operating surgeon utters. It records the sound being made around the operating surgeon, too.

b. Microphones on the Assistants

The microphones record the voice and speech the assistants utter and the sound being made around them.

c. Microphone on the Anesthesiologist

This microphone records the voice and speech the anesthesiologist utters and the sound being made around him or her.

d. Microphones on the Nurses

These are microphones that record the voice and speech the nurses utter and the sound being made around them.

(5) Devices of the Fifth Category

a. Door-Monitoring Sensor

This sensor monitors the opening and closing of the door to the operation room. It may be an opening/closing detecting switch, a monitor camera or the like.

b. IC Card Reader

The reader scans the ID card any person entering the operation room.

(6) Devices of the Sixth Category

a. Sensors for Anesthesia Monitors

These sensors monitors detect the heat beats, the partial pressure of oxygen, the body temperature, and the like.

b. BIS Monitor

The sensor detects the bispectral index (BIS) that represents the effect of the anesthetic administered to the patient.

c. Cranial Nerve Monitor

This monitor detects the brain waves and the like.

(7) Devices of the Seventh Category

a. Finger Print Sensor

This sensor reads the patient's fingerprints.

b. Iris Sensor

The sensor scans the patient's iris.

c. Electronic Clinical Record System

The system acquires the patient's clinical record from the electronic chart system.

d. Wrist Band Monitor

This monitor reads the ID data from the wristband that the patient wears.

As shown in FIG. 1, sixteen data-acquiring devices 9 are arranged in the operation room. Hereinafter, these data-acquiring devices 9 will be referred to as devices 9-1 to 9-16, whenever necessary to distinguish one from any other.

The data-acquiring device 9-1 is a sensor that monitors the BIS monitor 2. The data-acquiring device 9-2 is a sensor that monitors the power supply to the patient monitor 3. The data-acquiring devices 9-3, 9-15 and 9-16 are wide-angle cameras that capture moving picture of the interior of the operation room. The data-acquiring device 9-4 is a microphone that catches the sound and speech the anesthesiologist utters. The data-acquiring device 9-5 is a microphone that collects the sound and speech the surgeon utters. The data-acquiring device 9-6 is a microphone that catches the sound and speech the patient utters. The data-acquiring device 9-7 is a camera that captures moving picture of the region being treated. The data-acquiring devices 9-8, 9-9 and 9-14 are microphones that collect sounds made in the operation room. The data-acquiring device 9-10 is a sensor that monitors the power supply to the bipolar forceps 4. The data-acquiring device 9-11 is a camera captures moving picture of the region from the ceiling to provide a bird's-eye view. The data-acquiring device 9-12 is a sensor that detects the amount of contrast medium that the contrast-medium administering apparatus 5 has administered to the patient. The data-acquiring device 9-13 is a sensor that detects the state in which the suction catheter 6 is operating.

The data-acquiring devices 9 are connected to discrimination devices 10, respectively. This means that sixteen discrimination devices 10 are arranged in the operation room shown in FIG. 1. Hereinafter, the discrimination devices 10 will be referred to as devices 10-1, 10-2, . . . and 10-16, whenever necessary to distinguish one from any other. The discrimination devices 10-1 to 10-16 are connected to the medical information system 1.

Each discrimination device 10 receives medical data from the data-acquiring device 9 to which it is connected, and supplies the medical data to the medical information system 1. Each discrimination device 10 sends the ID data item that represents the type of the data-acquiring devices 9, to which it is connected, to the medical information system 1. Further, each discrimination device 10 stores the data representing the conditions in which to acquire medical data, and supplies this data to the data-acquiring device 9.

The X-ray imaging apparatus 7 applies X rays to the patient, acquiring medical images about the patient. The apparatus 7 has the function of outputting tag data items, in addition to the data representing the medical images. The tag data items the X-ray imaging apparatus 7 outputs represent, for example, the dose of X rays applied, the angles of the arm (to X and Y axes) and the region being examined. The tag data items are therefore part of the medical data.

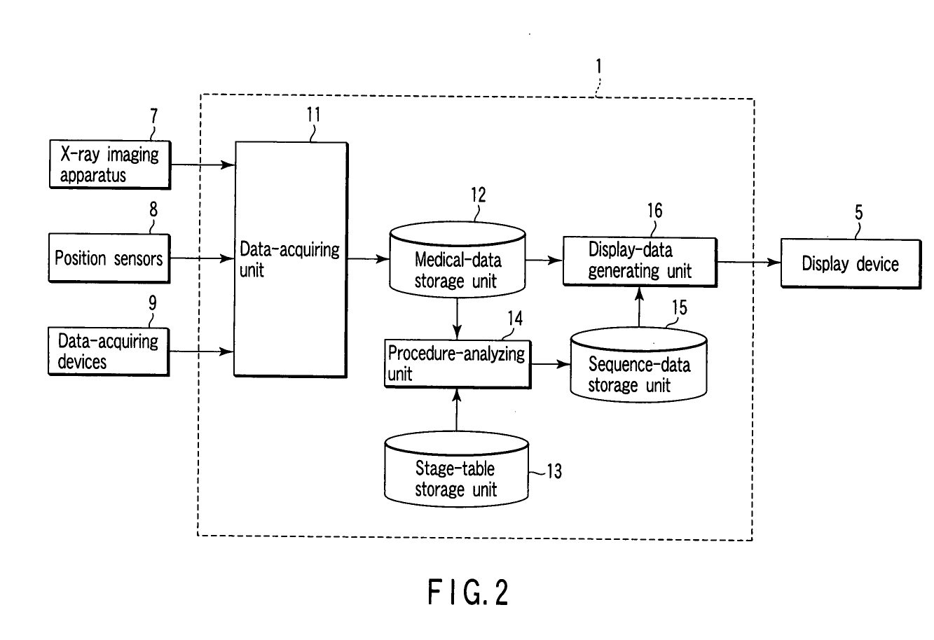

FIG. 2 is a block diagram illustrating the configuration of the medical information system 1.

As FIG. 2 shows, the medical information system 1 includes a data-acquiring unit 11, a medical-data storage unit 12, a stage-table storage unit 13, a procedure-analyzing unit 14, a sequence-data storage unit 15, and a display-data generating unit 16.

The medial data system 1 can have a general-purpose server and a computer, as basic hardware items. The data-acquiring unit 11, procedure-analyzing unit 14 and display-data generating unit 16 can be implemented as the processors incorporated in the server or the computer executes programs. These programs may be installed in the server or the computer beforehand. Alternatively, the programs may be distributed via a network or in the form of a removable recording medium, such as a magnetic disc, a magneto-optical disc, an optical disc or a semiconductor memory, and may then be installed, when necessary, in the server or the computer.

The units 11, 12, 13, 14, 15 and 16 may be implemented, either in part or in entirely, by hardware such as logic circuits. Alternatively, they may be implemented by a combination of hardware and software. The medical-data storage unit 12, stage-table storage unit 13 and sequence-data storage unit 15 can be storage devices incorporated in the server or the computer such as memories or hard disk drives, external storage devices connected to the server or the computer such as memories or hard disk drives, or removable recording media such as a magnetic disc, a magneto-optical disc or an optical disc.

The data-acquiring unit 11 acquires, as medical data, the outputs of the position sensors 8 and data-acquiring devices 9. The data-acquiring unit 11 writes the medical data items thus acquired, in the medical-data storage unit 12, labeling the time of acquisition to each data item.

The medical-data storage unit 12 stores the medical data it has received from the data-acquiring unit 11.

The stage-table storage unit 13 stores a stage table. The stage table is a data table describing conditions for determining which stage of the procedure is being performed.

The procedure-analyzing unit 14 analyzes the tag data items stored in the medical-data storage unit 12 stored in the stage-table storage unit 13, determining in what sequence the medical procedure has been performed, stage by stage. The unit 14 then generates the sequence data that represents the sequence determined.

The sequence-data storage unit 15 stores the sequence data the procedure-analyzing unit 14 has generated.

The display-data generating unit 16 utilizes the sequence data and the medical data stored medical-data storage unit 12, generating display data that represents the sequence of the medical procedure performed. The display data is supplied to a display device 5, which displays the sequence of stages performed in the medical procedure.

The configuration of the medical information system according to this embodiment has been described. The embodiment will be described in more detail.

### First Embodiment

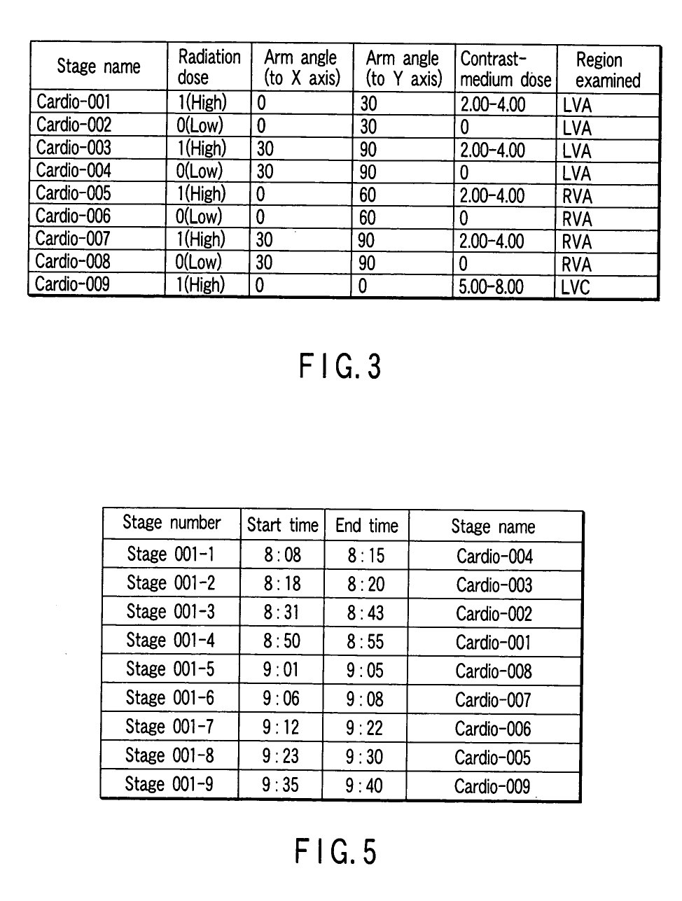

In the first embodiment, the stage table is such a data table as illustrated in FIG. 3. This stage table describes the various conditions for each stage, in association with the name of the stage to be carried out in the above-mentioned medical procedure. More precisely, the dose of X rays and the arm angle (to X and Y axes) for the X-ray imaging apparatus 7, the name of the region examined, and the dose of contrast medium for the contrast-medium administering apparatus 5 are described for each stage as shown in FIG. 3.

It will be described how the medical information system 1 operates, which is the first embodiment.

During a medical procedure is performed, the data-acquiring unit 11 receives medical data, i.e., all outputs of the X-ray imaging apparatus 7, position sensors 8 and data-acquiring devices 9. The unit 11 then writes the medical data in the medical-data storage unit 12. More specifically, the data-acquiring unit 11 starts acquiring the medical data when the lamps provided in the operation room, for example, are turned on. The unit 11 keeps acquiring the medical data until the lamps are turned off. The unit 11 acquires some data items at regular intervals. These data items include at least the tag data of the X-ray imaging apparatus 7 and the data representing the dose of the contrast medium. The intervals may have various values. Nonetheless, the intervals are one minute in the present embodiment.

Thus, various medical data items about the events that take place during the medical procedure are stored in the medical-data storage unit 12. In the medical-data storage unit 12, each medical data item is associated with the data item that represents the time when it is acquired. The medical-data storage unit 12 stores the data items concerning a medical procedure, in the form of a data file.

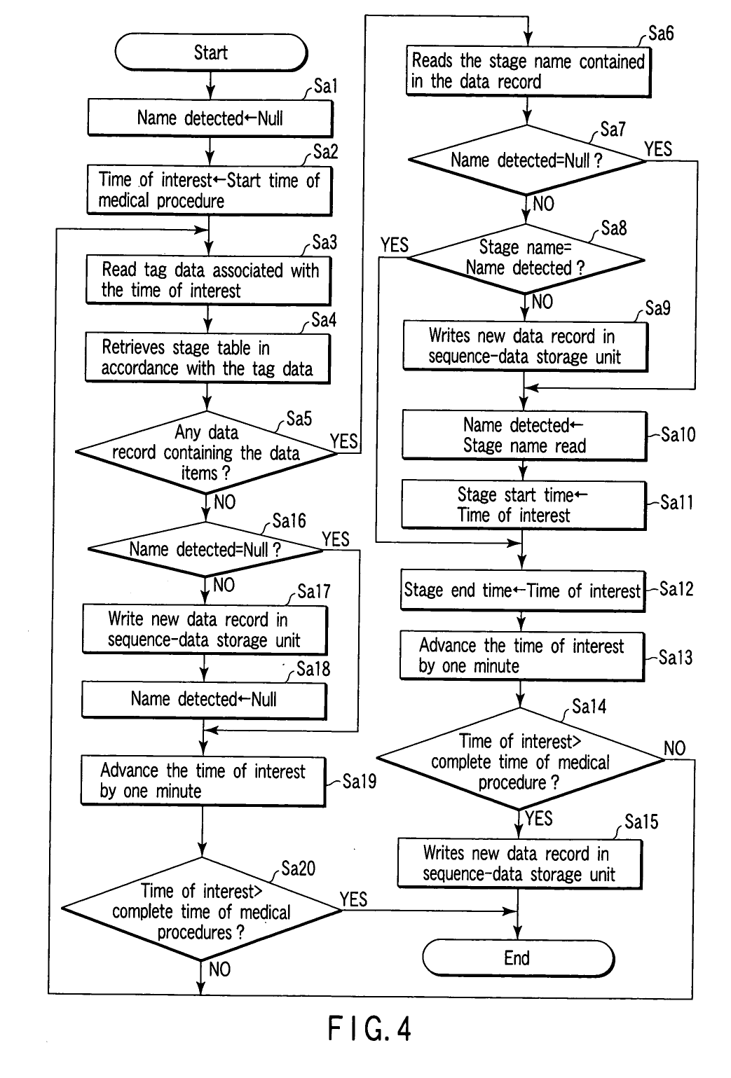

The procedure-analyzing unit 14 generates sequence data about a medical procedure already performed, as will be explained with reference to FIG. 4. More precisely, the procedure-analyzing unit 14 generates sequence data about one of the data files stored in the medical-data storage unit 12. The procedure-analyzing unit 14 may generates, at a time, the sequence data about all data files stored in the medical-data storage unit 12, or may generate the sequence data about only the data file designated by the operator.

In Step Sa1, the procedure-analyzing unit 14 substitutes “Null” for a variable called “name detected.” In Step Sa2, the procedure-analyzing unit 14 substitutes the procedure start time for a variable called “time of interest.”

In Step Sa3, the procedure-analyzing unit 14 reads from the medical-data storage unit 12 the tag data associated with the time of interest. More specifically, the procedure-analyzing unit 14 reads the medical data contains the X-ray dose, the arm angle (to X axis), the arm angle (to Y axis), the contrast-medium dose and the name of the region examined, which are contained in the medical data in the present embodiment. In Step Sa4, the procedure-analyzing unit 14 retrieves, from the stage table, the data record that is a combination the data items read in Step Sa3. In Step Sa5, the procedure-analyzing unit 14 determines whether each data record contains said combination of data items.

If YES in Step Sa5, the procedure-analyzing unit 14 goes to Step Sa6. In Step Sa6, the procedure-analyzing unit 14 reads the stage name that is contained in the data record retrieved in Step Sa4. If the data read in Step Sa3 represents the X-ray dose of 1 (high), the arm angle (to X axis) of 0, the arm angle (to Y axis) of 30, the contrast-medium dose of 2.00 to 4.00, the name of the region examined, LVA, the procedure-analyzing unit 14 reads “Cardio-001” as stage name in Step Sa6. The stage performed at the time of interest will then be designated.

In Step Sa7, the procedure-analyzing unit 14 determines whether the name detected is “Null” or not. Since “Null” has been set in Step Sa1 as name detected, the decision that the procedure-analyzing unit 14 makes in Step Sa7 is YES. As will be described later, the name detected is set as “Null” if no stages are designated one minute before the time of interest. Hence, the decision the procedure-analyzing unit 14 makes in Step Sa7 is YES when any stage is designated anew. If YES in Step Sa7, the procedure-analyzing unit 14 goes to Step Sa10. In Step Sa10, the procedure-analyzing unit 14 updates the name detected, to the stage name read in Step Sa6. Then, in Step Sa11, the procedure-analyzing unit 14 substitutes the time of interest for a variable called “stage start time.”

In Step Sa12, the procedure-analyzing unit 14 substitutes the time of interest for a variable called “stage end time.” The stage end time is the variable that indicates the time when one stage is completed. Although it is not detected that the stage has been completed, the stage designated now has been completed before the time of interest. Therefore, the time of interest is set as provisional stage end time. In Step Sa13, the procedure-analyzing unit 14 makes the time of interest advance by one minute. In Step Sa14, the procedure-analyzing unit 14 determines whether the time of interest, thus updated, follows the time when the medical procedure is completed. If NO in Step Sa14, the procedure-analyzing unit 14 returns to Step Sa3.

After substituting the stage name for the name detected, in Step Sa10, the procedure-analyzing unit 14 may goes to Step Sa7 again. If this is the case, the decision the procedure-analyzing unit 14 makes in Step Sa7 is NO. Then, the procedure-analyzing unit 14 goes to Step Sa8. In Step Sa8, the procedure-analyzing unit 14 determines whether the stage name read anew in Step Sa6 is identical to the name detected.

Assume that the stage name read anew in Step Sa6 differs from the name detected. This means that the stage designated ended one minute before the time of interest and that the stage designated for the time of interest started instead. Thus, if NO in Step Sa8, the procedure-analyzing unit 14 goes to Step Sa9. In Step Sa9, the procedure-analyzing unit 14 writes a new data record about the stage completed, in the sequence-data storage unit 15. The new data record is generated by describing the stage start time, stage end time and the name detected, in association with the serial stage number that indicates the writing order into the sequence-data storage unit 15. The procedure-analyzing unit 14 then goes to Step Sa10 in order to change the name of the new stage started to the name detected, and to change the time of interest to the stage start time. Thereafter, the procedure-analyzing unit 14 performs the steps that follow Steps S10, as described above.

If the stage name read anew in Step Sa6 is identical to the name detected, the stage designated one minute before the time of interest still goes on at the time of interest. Hence, if YES in Step Sa8, the procedure-analyzing unit 14 jumps to Step Sa12, skipping Steps Sa9 to Sa10. The unit 14 then performs Step Sa12 and some other following steps, as described above.

No records may be retrieved in Step Sa4. This means that no stages are carried out at the time of interest. The procedure-analyzing unit 14, which makes a negative decision (NO) in Step Sa5, goes to Step Sa16. In Step Sa16, the procedure-analyzing unit 14 determines whether the name detected is “Null” or not. If any stage is designated one minute before the time of interest, the name detected is not “Null.” That is, if the name detected is not “Null,” the stage designated one minute before the time of interest has been completed and no other stage has been started. Thus, the procedure-analyzing unit 14, which makes a negative decision (NO) in Step Sa16, goes to Step Sa17 and writes the new data record about the stage completed, in the sequence-data storage unit 15, in the same way as in Step Sa9.

In Step Sa18, the procedure-analyzing unit 14 substitutes “Null” for the name detected, because it could not designate any stage for the time of interest. Then, the procedure-analyzing unit 14 goes to Step Sa19.

The name detected may be determined to be “Null” in Step Sa16. In this case, all stages remain to be designated. Thus, if YES in Step Sa16, the procedure-analyzing unit 14 jumps to Step Sa19, skipping Steps Sa17 and Sa18.

In Step Sa19, the procedure-analyzing unit 14 makes the time of interest advance by one minute. In Step Sa20, the procedure-analyzing unit 14 determines whether the time of interest, thus updated, follows the time the medical procedure is completed. If NO in Step Sa20, the procedure-analyzing unit 14 returns to Step Sa3.

After the time of interest is advanced one minute in Step Sa13 or Sa19, a stage is designated on the basis of all tag data items acquired during one medical procedure, when the time of interest follows the time when the medical procedure is completed. Thus, if YES in Step Sa14, the procedure-analyzing unit 14 goes to Step Sa15. In Step Sa15, the procedure-analyzing unit 14 writes the new data record about the stage completed, in the sequence-data storage unit 15 in the same way as in Step Sa9. Thus, the process of FIG. 4 is terminated. If YES in Step Sa20, no stages are designated, and no data need to be written into the sequence-data storage unit 15. Hence, the procedure-analyzing unit 14 does not perform Step Sa15 and terminates the process of FIG. 4.

Thus, such sequence data as shown in FIG. 5, is generated in the sequence-data storage unit 15. As FIG. 5 shows, the sequence data consists of the names of the stages performed in the medical procedure, the types of thereof, the start time of each stage, and the end time of each stage, which are arranged in the order the stages were carried out.

The display-data generating unit 16 generates display data representing the sequence in which the stages of the medical procedure have been performed. The display data is supplied to the display device 5. The display device 5 displays a presenting image showing the sequence of stages that is represented by the display data.

Since the display device 5 displays the presenting image showing the sequence of stages (see FIG. 5), the operator can easily understand the sequence of the medical procedure.

The presenting image displayed by the display device 5 need not be identical the medical data stored in the medical-data storage unit 12 or to the sequence data stored in the sequence-data storage unit 15. Rather, it may be a combination of the medical data and the sequence data. If so, the operator can more easily grasp the medical procedure.

Assume that an image of the region in which significant events may occur in each stage may be automatically selected and displayed. Then, it is easy for the operator to confirm that stages have been performed in the medical procedure.

The surgeon may be startled or shaken while performing the medical procedure. Such psychological fluctuations, if any, are counted on the basis of the vital data about the surgeon. The number of psychological fluctuations may be displayed in association with the state name. Then, it can be determined in which errors may likely be made.

Moreover, the contrast-medium dose, the time spent in manipulating catheters, the number of times the critical catheter handing is performed, and the like may be displayed in association with the state name. These data items help to understand the degree of difficulty of the medical procedure. Note that these data items can be acquired from moving pictures captured by cameras.

### Second Embodiment

In a second embodiment of this invention, the stage table describes the condition for determining whether a stage is performed, the condition for starting the stage and the condition for ending the stage, in association with the name of the stage. These conditions indicate the state of an event or the states of events. Some of these events relate to the operating conditions of the medical apparatuses. The remaining events relate to the positions of the medical staff, the positions of the medical apparatuses, the positions of the medical instruments and the positions of the medical articles.

The conditions concerning, for example, the stage of stopping bleeding are as follows:

a. Condition for Determining the Occurrence

The stage is considered to occur when the operating surgeon held the bipolar forceps 4 and power was supplied to the forceps 4 set in the coagulation mode.

b. Condition for Determining the Start

Assume that the forceps 4 in the coagulation mode repeatedly received power, at intervals of 30 seconds or less. It is determined whether the suction catheter 6 was used or sterilized gauze was inserted into the patient at most one minute before the start of the first supply of power. If YES, it is determined whether the catheter 6 was used or sterilized gauze was inserted, at most one minute before the use of the catheter 6 or the insertion of gauze thus determined. If YES, similar determination is repeated until it is determined that the catheter 6 was used or sterilized gauze was inserted for the first time. The time when the catheter 6 was used or gauze was inserted for the first time is regarded as the time when the stage of stopping bleeding was started.

c. Condition for Determining the End

Also, assume that the forceps 4 in the coagulation mode repeatedly received power, at intervals of 30 seconds or less. It is determined whether the suction catheter 6 was used or sterilized gauze was inserted into the patient at most one minute after the end of the last supply of power. If YES, it is determined whether the catheter 6 was used or sterilized gauze was inserted, at most one minute after the use of the catheter 6 or the insertion of gauze thus determined. If YES, similar determination is repeated until it is determined that the catheter 6 was used or sterilized gauze was inserted for the last time. The time when the catheter 6 was used or gauze was inserted for the last time is regarded as the time when the stage of stopping bleeding was ended.

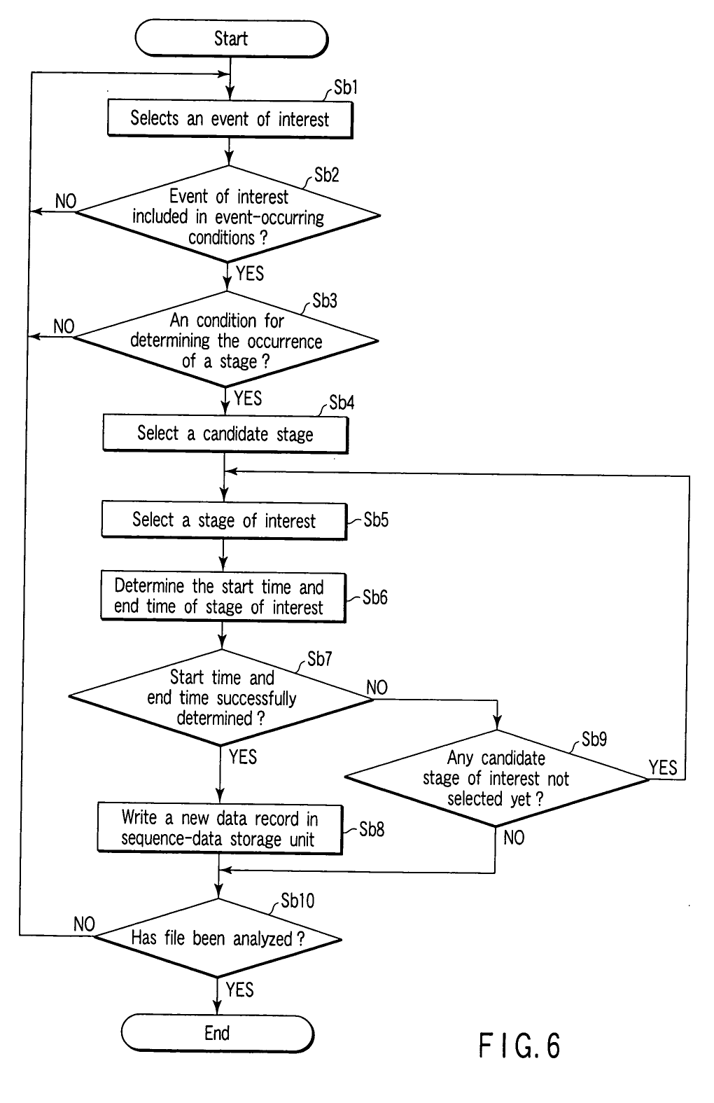

The procedure-analyzing unit 14 generates sequence data about a medical procedure already performed, as will be explained with reference to FIG. 6. More precisely, the procedure-analyzing unit 14 generates sequence data about one of the data files stored in the medical-data storage unit 12. The procedure-analyzing unit 14 may generates, at a time, the sequence data about all data files stored in the medical-data storage unit 12, or may generate the sequence data about only the data file designated by the operator.

In Step Sb1, the procedure-analyzing unit 14 selects one of the events recorded, as event of interest. Step Sb1 is repeatedly performed as will be described later. Nonetheless, the procedure-analyzing is unit 14 selects events, each as event of interest, one by one in the order they occurred.

In Step Sb2, the procedure-analyzing unit 14 determines whether the event of interest is included in the conditions described in the stage table, i.e., conditions in which events may occur. If NO, the procedure-analyzing unit 14 returns to Step Sb1 and selects another event as event of interest. Then, the procedure-analyzing unit 14 performs Step Sb2 again. If YES in Step Sb2, the procedure-analyzing unit 14 goes to Step Sb3. The event of interest may be, for example, the moving of the bipolar forceps 4. In this case, it is possible that the operating surgeon held the forceps 4. Thus, the decision the procedure-analyzing unit 14 makes in Step Sb2 is affirmative (YES), because the event of interest is included in the conditions for determining the occurrence of a stage of stopping bleeding.

In Step Sb3, the procedure-analyzing unit 14 determines whether the condition for determining the occurrence of the stages including the event of interest is established. If NO, the procedure-analyzing unit 14 returns to Step Sb1. In Step Sb1, the procedure-analyzing unit 14 selects another event as event of interest. Then, the procedure-analyzing unit 14 performs Step Sb2 again. If YES in Step Sb3, the procedure-analyzing unit 14 goes from Step Sb3 to Step Sb4. In the stage of stopping bleeding, the procedure-analyzing unit 14 makes a positive decision (YES) if the position of the bipolar forceps 4 and the position of the operating surgeon's hand are identical and if it is confirmed, from the other events, that the bipolar forceps 4 is set in the coagulation mode.

In Step Sb4, the procedure-analyzing unit 14 selects the stage that accords with the condition for determining the occurrence, as a candidate stage. If there are two or more conditions for determining the occurrence, the procedure-analyzing unit 14 selects all stages that accord with these conditions, as candidate stages. In Step Sb5, the procedure-analyzing unit 14 selects one of the candidate stages, as stage of interest. In Step Sb6, the procedure-analyzing unit 14 tries to determine the start time and end time of the stage of interest, from the conditions for starting and ending the stage of interest.

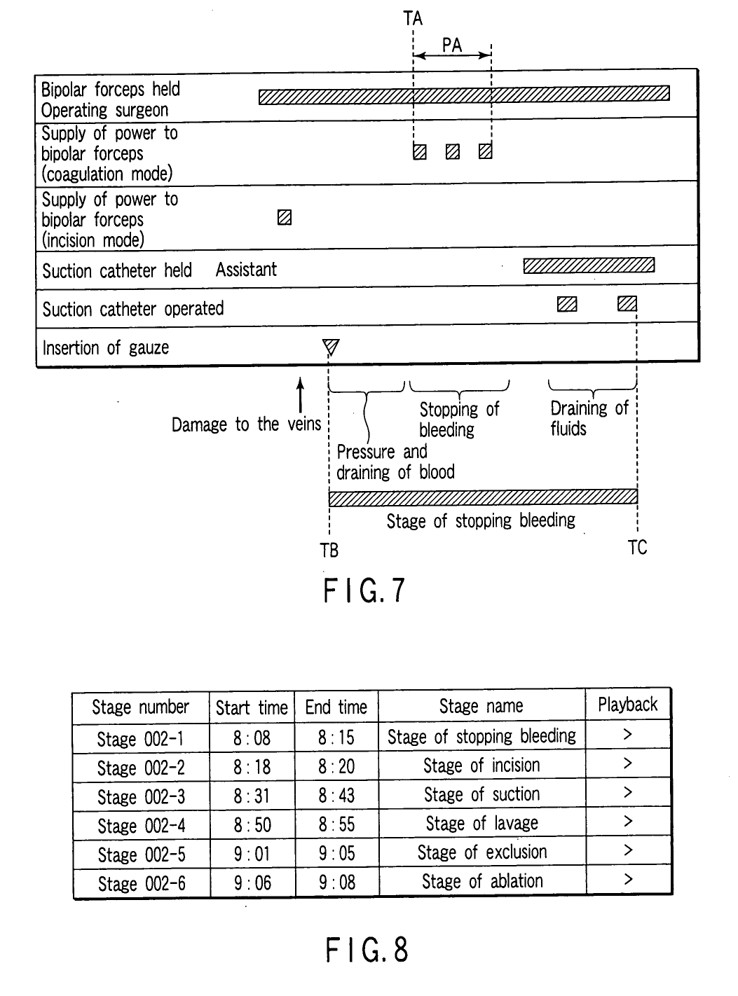

In the state illustrated in FIG. 7, for example, it is determined at time TA that the stage of stopping bleeding has occurred. Assume that the bipolar forceps 4 in the coagulation mode repeatedly received power for a period PA, at intervals of 30 seconds or less. Then, it can be determined that the stage of stopping bleeding started at time TB that preceded the period PA and ended at time TC that follows the period PA.

In Step Sb7, the procedure-analyzing unit 14 determines whether the start time and end time have been successfully determined. If YES, the procedure-analyzing unit 14 goes from Step Sb7 to Step Sb8. In Step Sb8, the procedure-analyzing unit 14 writes a new data record about the stage of interest, in the sequence-data storage unit 15. In other words, the unit 14 determines that the stage of interest has been performed. The new data record describes the stage start time, stage end time and stage name that are recorded in association with the serial stage number. The stage start time and the stage end time correspond to those that have been determined in Step Sb6. The stage name pertains to the stage that is regarded as the stage of interest at present. The procedure-analyzing unit 14 then goes to Step Sb10.

If NO in Step Sb7, the procedure-analyzing unit 14 goes to Step Sb9. In Step Sb9, the procedure-analyzing unit 14 determines whether the candidate stages selected in Step Sb4 include a stage that has not been selected as stage of interest. If YES, the procedure-analyzing unit 14 returns to Step Sb5 and changes the stage of interest to another. The unit 14 then repeats Steps Sb6, Sb7, Sb8 and Sb9. If NO in Step Sb9, the procedure-analyzing unit 14 goes to Step Sb10.

In Step Sb10, the procedure-analyzing unit 14 determines whether the file being processed has been analyzed. If NO, the procedure-analyzing unit 14 returns to Step Sb1. In Step Sb1, the procedure-analyzing unit 14 selects another event as event of interest. Then, the procedure-analyzing unit 14 repeats the subsequent steps. If YES in Step Sb10, the procedure-analyzing unit 14 terminates the process of FIG. 6.

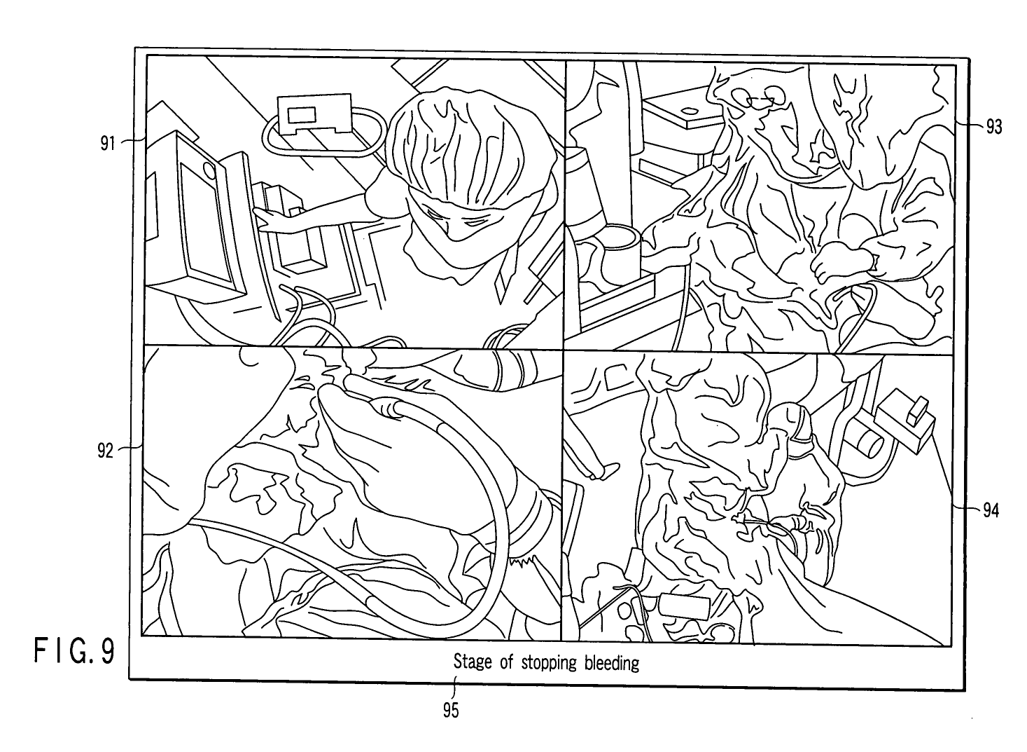

In the second embodiment, the display-data generating unit 16 generates display data representing such a presenting image as shown in FIG. 8. From the presenting image displayed, the operator can easily understand the sequence of the medical procedure that has been performed. The presenting image includes playback buttons “>.” When the operator clicks any one of the playback buttons, the display-data generating unit 16 reads from the medical-data storage unit 12 the video data generated during the stage associated with the playback button clicked. From the video data, the unit 16 then generates display data. The display data generated contains a data item that represents the stage name. Hence, the display device 5 can display the stage name, together with the video images. FIG. 9 shows some images displayed on the display device 5 on the basis of the display data. The images shown in FIG. 9 include video images 91 to 94 that are displayed simultaneously. The video image 91 is captured by a camera provided under a shadow-less light. The video image 92 is captured by a camera provided beside a surgical microscope. The video image 93 is captured by a camera provided at a medical apparatus. The video image 94 is captured by a camera provided on the ceiling of an operating room. The images shown in FIG. 9 also include an image 95 representing the stage name as well as these video images. This makes it easy for the operator to know which stage the images being played back pertain to.

Thus, the second embodiment can analyze the sequence of the medical procedure on the basis of not only the operation conditions of the medical apparatuses, but also the positions of the medical staff and medical apparatuses. The stages of the medical procedure can be well distinguished form each other. This is because the events occurring in each stag, such as the surgeon's holding the bipolar forceps 4 and the insertion of gauze into the patient, can be discriminated, not as in the case where the sequence of the medical procedure is analyzed from only the operation conditions of the medical apparatuses.

The embodiments described above can be modified in various manners as will be described below.

The first embodiment acquires data from medical apparatuses such as the X-ray imaging apparatus 7 and the contrast-medium administering apparatus 5. Nonetheless, it may be designed to acquire data from some other medical apparatuses. In this case, it suffices to use stage tables that accord with the characteristics of the additional medical apparatuses.

Further, the procedure-analyzing unit 14 may generate, in real time, the sequence data from the data input to the data-acquiring unit 11, while the medical procedure is being performed.

In each embodiment described above, only the stage start time may be determined. Namely, the stage end time need not be determined.

In each embodiment, the time when the medical data is acquired is regarded as the time when an event related to the data takes place. Instead, the time data may be acquired, together with the medical data, from the X-ray imaging apparatus 7, the position sensors 8 or the data-acquiring devices 9.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical information system for processing data to manage a medical procedures that includes a plurality of stages each of which is a combination of evens, said system comprising:
a data-acquiring unit which acquires, in time sequence, condition data representing at least one of conditions of medical apparatuses used in the medical procedure, conditions of medical instruments used in the medical procedure, conditions of medical articles used in the medical procedure, conditions of patient and conditions of medical staff performing the medical procedure;
a first determining unit which determines an order in which the stages are performed, from a condition which is determined on the basis of the condition data and in which the events occur;
a second determining unit which determines a start timing of each of the stages on the basis of the condition data; and
a unit which generates sequence data representing the order and the start timing of each stage.

2. The medical information system according to claim 1, wherein the data-acquiring unit acquires at least one data item of data representing operating conditions of the medical apparatuses, data representing positions of the medical staff, data representing positions of the medical apparatuses, data representing positions of the medical instruments and data representing positions of the medical articles.

3. The medical information system according to claim 2, wherein the data-acquiring unit acquires data representing a radiation dose, as the data representing the operating conditions of the medical apparatuses.

4. The medical information system according to claim 2, wherein the first determining unit determines the condition in which the events occur, on the basis of at least one of changes in the operating conditions of the medical apparatuses, changes in the positions of the medical staff, changes in the positions of the medical apparatuses, changes in the positions of the medical instruments and changes in the positions of the medical articles, and determines the order from relation that the events have with one another.

5. The medical information system according to claim 4, wherein the first determining unit determines that the stage has started, when the operating conditions of the medical apparatus accord with conditions predetermined for the stage.

6. The medical information system according to claim 4, wherein the first determining unit determines that the stage has started, when the conditions in which the events occur accord with conditions preset for the stage.

7. The medical information system according to claim 1, wherein the second determining unit determines an end timing of each stage on the basis of the condition data.

8. The medical information system according to claim 1, further comprising a generating unit which generates display data representing the order, from the sequence data.

9. The medical information system according to claim 8, which is configured to access a storage medium that stores timing data together with medical data about the medical procedure already performed, and in which the generating unit determines which stage the medical data pertains to, on the basis of the start timing of each stage and the timing data, and generates the display data that represents the relation between the medical data and the stage.

10. The medical information system according to claim 9, wherein the medical data includes at leas one item selected from the group consisting of images imaged by the medical apparatuses, a contrast-medium dose administered, time spent in manipulating catheters, number of times the catheters were improperly handled, and number of psychological fluctuations the surgeon had during the medical procedure.

11. A program product for causing a computer to function as a medical information system designed to process data to manage medical procedures that include a plurality of stages each of which is a combination of evens, said system comprising:
a data-acquiring unit which acquires, in time sequence, condition data representing at least one of conditions of medical apparatuses used in the medical procedure, conditions of medical instruments used in the medical procedure, conditions of medical articles used in the medical procedure, conditions of patient and conditions of medical staff performing the medical procedure;
a first determining unit which determines an order in which the stages are performed, from a condition which is determined on the basis of the condition data and in which the events occur;
a second determining unit which determines a start timing of each of the stages on the basis of the condition data; and
a unit which generates sequence data representing the order and the start timing of each stage.

12. The program product according to claim 11, which causes the computer to make the second determining unit determines an end timing of each stage on the basis of the condition data, too.

13. The program product according to claim 11, which causes the computer to function as a generating unit which generates display data representing the order, from the sequence data.

14. The program product according to claim 13, which causes the computer to access a storage medium that stores timing data together with medical data about the medical procedure already performed, and causes the generating unit to determine which stage the medical data pertains to, on the basis of the start timing of each stage and the timing data stored in the storage medium, and generates the display data that represents the relation between the medical data and the stage.

15. A method of processing data to manage a medical procedures that includes a plurality of stages each of which is a combination of evens, said method comprising:
acquiring, in time sequence, condition data representing at least one of conditions of medical apparatuses used in the medical procedure, conditions of medical instruments used in the medical procedure, conditions of medical articles used in the medical procedure, conditions of patient and conditions of medical staff performing the medical procedure;
determining an order in which the stages are performed, from a condition which is determined on the basis of the condition data and in which the events occur;
determining a start timing of each of the stages on the basis of the condition data; and
generating sequence data representing the order and the start timing of each stage.

16. The method according to claim 15, wherein an end timing of each stage on the basis of the condition data, along with the start timing of each stage.

17. The method according to claim 15, wherein display data representing the order is generated from the sequence data.

18. The method according to claim 17, wherein a storage medium that stores timing data together with medical data about the medical procedure already performed is accessed, it is determined which stage the medical data pertains to, on the-basis of the start timing of each stage and the timing data, and the display data representing the relation between the medical data and the stage is generated.
